(19) United States
(12) Patent Application Publication
(10) Pub. No.: US 2011/0144513 A1
(43) Pub. Date: Jun. 16, 2011

(54) METHODS FOR IMPROVED ANALYSIS OF HEART RATE VARIABILITY

(76) Inventor: Ryan M. Deluz, Encinitas, CA (US)

(21) Appl. No.: 12/969,521

(22) Filed: Dec. 15, 2010

Related U.S. Application Data
(60) Provisional application No. 61/286,503, filed on Dec. 15, 2009.

(51) Int. Cl. A61B 5/0402

(52) U.S. Cl. 600/519

(57) ABSTRACT

Methods for analysis of heart rate variability can be used in gaming and other software applications as disclosed herein; wherein improvements include a faster analysis of heart rate variability, prediction of future values, and assessment of value deviations, among others. The described methods interpolate variations in heart rate variability using one or more of the factors: LF amplitude factor (kALF), HF amplitude factor (kAHF), and deviation factor (kdev). Other features of the improved methods are described in detail herein.

### FIELD OF THE INVENTION

The present invention relates generally to methods for analysis of heart rate variability, and more specifically to methods for analysis of heart rate variability for use in computerized software applications.

### BACKGROUND OF THE INVENTION

The heart rate frequency (fh) is not a constant frequency, rather it is subject to natural variations between beats. This change of heart rate is referred to as the heart rate variability (HRV). To determine HRV, the heartbeats supplied per unit of time by an electrocardiograph, or other pulse-measuring instrument, are usually interpolated for a predefined sampling period (Ts), producing a heart rate value for each multiple of the sampling period.

Studies show that HRV may be used as an index of physical, emotional and/or mental arousal or health. Changes in a person's breathing pattern, level of stress, and other physical attributes typically correspond to variations in the heart rate. Biofeedback training with HRV can be used for relaxation or peak performance training.

Standard methods of HRV analysis often involve measuring heart rate frequencies by determining the ratio between the low frequency (LF) amplitudes and the high frequency (HF) amplitudes. Generally, LF amplitudes are those defined as from 0.04 Hz to 0.15 Hz, and HF amplitudes are those defined as from 0.15 Hz to 0.4 Hz. The LF/HF ratio is used as an indicator of stress level, autonomic balance, breathing and/or emotional state.

In biofeedback training, this LF/HF ratio along with a graph showing heart rate changes, is displayed to a user to enable him or her to track changes in vital functions (i.e., breathing and heart rate), and use this information to adjust the amplitude of various frequencies (increasing target frequencies and lowering non-desired frequencies). Biofeedback gaming applications also make use of the LF/HF ratio, as where the character on screen could move faster, improve his health, or have other positive responses when LF rises in comparison with the HF. This can be used for positive physical, emotional or mental improvements, or simply to make game play more enjoyable.

The specific frequencies used can vary, as one can compare any target frequency, which we will refer to herein as LF, to any other set of undesired frequencies hereinafter referred to here as HF. Some methods compare a target frequency to all other frequencies, or often even a total of all frequencies to include the target frequency.

Relevant prior art in the field of biofeedback that makes use of HRV frequency analysis relies mainly on determining the relative values of LF to HF, such as a LF/HF ratio. For example, as a user's heart rate is measured over a period of time, the heart rate will vary over this period due to factors such as inhaling and exhaling a breath of air, level of stress, and other physical factors. These heart rate variations are recorded by an electrocardiograph or similar instrument and plotted over the measured period, for example one minute. The plot of heart rate over a one minute interval will provide a wave-like plot. The value of LF changes with respect to the value of HF changes is measured using a bar graph or other indicator, such that a percentage or visual relationship between the LF/HF ratio is provided.

One problem with using a LF/HF ratio alone as an indicator of HRV is that the method does not provide very timely and accurate feedback to the user. HRV, as reflected in a LF/HF ratio, requires processing a significant number of consecutive heart beats, often 30 seconds to five minutes of data. Because of this, it can take 30 seconds or more to change the LF/HF ratio in a meaningful way. Because of the time lag, feedback to the user is delayed thus making it difficult for him to monitor the instantaneous effects of self-regulation. He cannot, for instance, tell when he has made a correct change in his breathing or relaxation until a substantial time has passed. Also, game play that integrates this method of HRV analysis is boring because game features are slow to respond. This has prevented commercial success of games that include HRV for training positive physical, mental or emotional improvements or for improved game play, including making the game more fun and interesting.

It is generally recognized that consistent LF variations in the heart rate with minimal HF influence (noise) is an indicator of a healthy HRV pattern. However, those of skill in the art will recognize that currently available methods of heart rate analysis utilize normalization factors to more or less weight a desired characteristic, such as LF variations, more heavily in the overall analysis, such that a dynamic plot can be achieved. Without a true and balanced representation of heart rate variability, certain applications cannot be effectively adapted for use with these methods. Therefore, prior art methods which utilize these normalization factors are insufficient for representing HRV in applications such as biofeedback.

The current state of the art is well defined in U.S. Pat. App. Pub. 2008/0058662, titled “METHOD FOR EVALUATING HEART RATE VARIABILITY”, hereinafter referred to as the '662 application; the entire contents of which are hereby incorporated by reference. The '662 application discloses some of the problems with prior art methods of HRV analysis in the field of biofeedback, however the '662 application solves these problems by simply reducing the weight of the HF value in the LF/HF ratio, ultimately providing a solution as follows:

$$H R {V}_{i} = {\tilde{I}}_{max , i} * f ( {\tilde{I}}_{ref , i} ) , \\ where ;$$

$$f ( {\tilde{I}}_{ref} ) = \frac{1}{{( {\tilde{I}}_{ref} )}^{a}} where 0 < α < 1.$$

Therefore, the '662 application provides a manipulation of the HRV=LF/HF equation known in the prior art, substituting instead a method for reducing the HF value by a normalization step using an exponent “a”, i.e. (0<a<1), to adjust weighting of the HF value, thereby increasing the weighting of LF in the overall analysis. Although this adjustment, or normalization of the HRV may yield a method of analysis compatible with biofeedback, the overall analysis of HRV remains largely unimproved and maintains the same problems as the prior art.

The prior art fails to teach a method of HRV analysis which: (i) provides immediate feedback to a user, such that the user can effectively react to biofeedback representations; (ii) accounts for heartbeat variations utilizing factors other than the value of LF with respect to HF; and (iii) provides interactive and dynamic HRV-driven game play.

The prior art methods are further constrained between a maximum and minimum value, i.e. 100% and 0%, respectively. These terminal ends present programming problems when used with biofeedback gaming applications.

Therefore, there is a longstanding need in the art for improved methods for the analysis of heart rate variability for use in computerized software applications including biofeedback and video game applications.

### SUMMARY OF THE INVENTION

It is therefore an object of the present invention to solve the aforementioned problems in the art by providing methods for heart rate variability analysis that are capable of: (i) immediate feedback response, allowing a user to effectively monitor his progress in self-regulation; (ii) providing an improved interactive, dynamic, and faster HRV-driven game application; and (iii) providing improved accuracy and feedback to the user by analyzing absolute LF and HF amplitudes, as well as changes in these frequencies, that would not otherwise be indicated by the LF/HF ratio.

In one embodiment, the method includes the steps of: (i) measuring heart rate using an electrocardiograph or similar instrument over a period of time; (ii) interpolating the heart rate measured over said period of time to yield an analysis of heart rate variation (HRV); (iii) determining the relative values of low frequency (LF) measurements, and high frequency (HF) measurements; (iv) adjusting the values of LF and HF measurements with one or more normalization factors including: LF amplitude factor (kALF), HF amplitude factor (kAHF), and deviations from a predicted value; and (v) providing a visualization of the adjusted proportions of the values of LF and HF, i.e. HRV=LFadj/HFadj.

Other embodiments of the invention will become apparent to one having skill in the art in view of the attached drawings and the detailed description.

### BRIEF DESCRIPTION OF THE DRAWINGS

FIG. 1a is spectrograph of an ideal HRV plot, where the heart rate varies from about 55 beats per minute to about 75 beats per minute, the HRV plot illustrates consistent LF variation without influence from HF noise.

FIG. 1b is a graphical representation of the value of LF compared to the value of HF variations in the heart rate spectrograph of FIG. 1a.

FIG. 2a is spectrograph of an HRV plot, where the heart rate varies from about 60 beats per minute to about 65 beats per minute, the HRV plot illustrates consistent HF variation or noise.

FIG. 2b is a graphical representation of the value of LF compared to the value of HF variations in the heart rate spectrograph of FIG. 2a.

FIG. 3a is spectrograph of an HRV plot, where the heart rate varies from about 55 beats per minute to about 75 beats per minute, the HRV plot illustrates consistent LF variation with consistent influence from HF variations.

FIG. 3b is a graphical representation of the value of LF compared to the value of HF variations in the heart rate spectrograph of FIG. 3a.

FIG. 4a is spectrograph of an HRV plot, where the heart rate varies from about 55 beats per minute (bpm) to about 75 beats per minute during the first 30 seconds of the period, and the heart rate varies from about 58 bpm to about 68 bpm in the last 30 seconds of the one minute period measured; the HRV plot illustrates consistent LF variation without influence from HF noise, however there is a decrease in the amplitude of LF variations over the second half of the measured period.

FIG. 4b is a graphical representation of the value of LF compared to the value of HF variations in the heart rate spectrograph of FIG. 4a during the first 30 seconds of the measured period.

FIG. 4c is a graphical representation of the value of LF compared to the value of HF variations in the heart rate spectrograph of FIG. 4a during the last 30 seconds of the measured period.

FIG. 5a is spectrograph of an HRV plot, where the heart rate varies from about 60 beats per minute to about 63 beats per minute, the HRV plot illustrates consistent LF variation without influence from HF noise, however the amplitude of LF variations is extremely low.

FIG. 5b is a graphical representation of the value of LF compared to the value of HF variations in the heart rate spectrograph of FIG. 5a.

FIG. 6a is spectrograph of an HRV plot, where the heart rate varies from about 55 beats per minute to about 75 beats per minute, the HRV plot illustrates consistent LF variation with consistent influence from HF noise, however the HF noise is lower in amplitude during the first 30 seconds of the measured period, while the amplitude of the HF noise is more significant in the last 30 seconds of the period measured.

FIG. 6b is a graphical representation of the value of LF compared to the value of HF variations in the heart rate spectrograph of FIG. 6a during the first 30 seconds of the measured period.

FIG. 6c is a graphical representation of the value of LF compared to the value of HF variations in the heart rate spectrograph of FIG. 6a during the last 30 seconds of the measured period.

FIG. 7a is spectrograph of an HRV plot, where the heart rate varies from about 55 beats per minute to about 85 beats per minute, the HRV plot illustrates consistent LF variation with influence from HF noise, however the HF amplitude is relatively insignificant.

FIG. 7b is a graphical representation of the value of LF compared to the value of HF variations in the heart rate spectrograph of FIG. 7a.

FIG. 8a is spectrograph of an HRV plot, where the heart rate varies from about 55 beats per minute to about 75 beats per minute, the HRV plot illustrates consistent LF variation with influence from HF noise at a relatively minimal amplitude during the first 30 seconds of the measured period; the HRV is relatively flat between second 31 and second 40, and the HRV is resumed at second 41 through the end of the measured interval where lower amplitude LF variations combine with high amplitude HF variations.

FIG. 8b is a graphical representation of the value of LF compared to the value of HF variations in the heart rate spectrograph of FIG. 8a during the first 30 seconds of the measured period.

FIG. 8c is a graphical representation of the value of LF compared to the value of HF variations in the heart rate spectrograph of FIG. 8a during the ten second period starting at second 31 and ending at second 40 of the one-minute period for measurement.

FIG. 8d is a graphical representation of the value of LF compared to the value of HF variations in the heart rate spectrograph of FIG. 8a during the twenty second period starting at second 41 and ending at second 60 of the one-minute period for measurement.

FIG. 9 is spectrograph of an HRV plot, where the heart rate varies from about 55 beats per minute to about 75 beats per minute, the HRV plot illustrates consistent LF variation without influence from HF noise; a second representation illustrates a predicted heart rate, where the predicted heart rate is graphically represented at 20 bpm below the measured heart rate; the measured heart rate deviates from the predicted heart rate at about 18 seconds into the measured period.

### DETAILED DESCRIPTION

The present invention provides improved methods for heart rate variability (HRV) analysis. The prior art methods discussed in the foregoing specification fail to contemplate changes in the amplitude associated with low frequency (LF) variations and high frequency (HF) variations in heart rate. Furthermore, the prior art fails to provide methods which predict subsequent values for heart rate analysis, and compare those predicted values with measured values to determine if a deviation is introduced into the heart rate pattern.

The inventor of the present invention recognized that prior art methods for heart rate variability (HRV) analysis are for the most part incompatible with biofeedback gaming applications. Furthermore, the improved methods for HRV analysis disclosed herein can be used to provide immediate feedback to a user for improved biofeedback interaction in a variety of applications, including biofeedback applications for clinical behavioral treatments, and enhanced video game interactivity.

As described above, currently available HRV analysis methods involve lengthy collections of data to interpolate relevant information to a user, and therefore these methods are insufficient for biofeedback applications due to the delay in time inherent in these methods. For example, currently available methods require about 30 seconds to determine the state of the user using an LF/HF ratio or similar method. Because heart rate changes during video game play, and while a user of a biofeedback therapeutic system, the changes are not reflected immediately under current methods of HRV analysis. Accordingly, the following methods seek to provide immediate representations of the status of a users level of stress, heart rate, and other physiological factors associated with HRV analysis.

Of particular use is the differentiation between high amplitude LF heart rate variations and low amplitude LF variations. The prior art methods couple all LF values into a single category, LF, whereas the certain embodiments of present methods differentiate relatively high amplitude LF variations in the heart rate from relatively low amplitude variations in the heart rate. The presently described methods further differentiate changes in LF variations over time.

Similarly, certain embodiments of the present invention differentiate relatively high amplitude HF variations in heart rate from relatively low amplitude HF variations in the heart rate. Additionally, the present methods further differentiate changes in HF variations over time.

Additionally, the present invention provides a method for predicting future values of the heart rate based on an interpretation, such as a trend, of prior or contemporaneous heart rate values. The predicted heart rate can be measured against the measured heart rate to evaluate the deviation therebetween. The inventor of the present invention has recognized that because consistent variations in the heart rate are a sign of a healthy relaxed state, deviations or inconsistencies with the norm are likely therefore to be a representation of an unhealthy state, such as stress, fatigue, or the like.

In a general embodiment of the present invention, a method of HRV analysis includes the steps of: (i) providing an electrocardiographic device; (ii) measuring a users heart rate over a period of time (Ts), where Ts is generally measured in seconds; (iii) interpolating the value of low frequency (LF) and high frequency (HF) variations in the heart rate using one or more normalization factors selected from: LF amplitude factor (kALF), HF amplitude factor (kAHF), and deviation factor (kdev); representing the proportion of adjusted LF variations to adjusted HF variations, i.e. HRV=LFadj/HFadj.

In another embodiment, changes in: LF/HF; LF amplitude; LF frequency; HF amplitude; HF frequency; or deviations from a trend value for heart rate are represented by a visual indicator, such as sound, visual display properties such as color, or gaming attributes such as character speed, or other character or environment representations. In this embodiment, a change in one of the heart rate variability indicators corresponds to a correlated change in a visual indicator, such that the user is immediately notified of his or her present relaxation state.

LF amplitude factor (kALF) is provided as a normalization factor to more adequately represent the value of LF over a measured period. For example, because the prior art collectively refers to low amplitude LF variations in heart rate and high amplitude LF variations in heart rate as “LF”, the value of LF is overly broad and is not distinguished from those very positive representations of a users HRV where the LF variation includes a high amplitude, and the relatively negative representations of a users state where the LF variation includes a low amplitude LF variation.

Because the overall interpretation of HRV can vary with respect to age and other individual factors, the amplitude of LF variations that are positive are somewhat subjective. Generally, an LF variation with an amplitude of 10 beats per minute is considered a positive indicator of low stress and strong health. In contrast, a general amplitude of 3 bpm or less in a particular LF variation can be a representation that a user is less healthy, especially where the same user frequently produces high amplitude LF variations on the order of about 10 beats per minute. The prior art fails to contemplate relative strengths of these signals, and merely combines all LF variations into what becomes a single positive indicator.

LF amplitude factor (kALF) is generally a normalized value between 0 and 1, which represents the strength of LF variations in the measured heart rate. Depending on the clinicians determination of a positive amplitude variation with respect to a particular users heart rate, the normalization factor can be optimized by adjusting the value of “k” and multiplying by the LF Amplitude (ALF). Various methods can determine the measured value of LF Amplitude (ALF), for example LF Amplitude (ALF) can equal the average amplitude of LF heart rate variations over a period of time, or alternatively LF Amplitude (ALF) can equal a mean amplitude of LF heart rate variations over a period of time. Numerous other representations of the LF Amplitude will be apparent to one having skill in the art, and are intended to be within the spirit and scope of the invention.

Similarly, HF amplitude factor (kAHF) is generally a normalized value between 0 and 1, which represents the strength of HF variations in the measured heart rate. Depending on the clinicians determination of a positive amplitude variation with respect to a particular users heart rate, the normalization factor can be optimized by adjusting the value of “k” and multiplying by the HF Amplitude (AHF). Various methods can determine the measured value of HF Amplitude (AHF), for example HF Amplitude (AHF) can equal the average amplitude of HF heart rate variations over a period of time, or alternatively HF Amplitude (AHF) can equal a mean amplitude of HF heart rate variations over a period of time. Numerous other representations of the HF Amplitude will be apparent to one having skill in the art, and are intended to be within the spirit and scope of the invention.

In one embodiment, an improved representation of HRV is illustrated by the following mathematical representation:

HRV=[(kALF)*LF]/[(kAHF)*HF].

In another embodiment, an improved representation of HRV is illustrated by the following mathematical representation:

HRV=[(kALF)*LF]/[HF].

In yet another embodiment, an improved representation of HRV is illustrated by the following mathematical representation:

HRV=[LF]/[(kAHF)*HF].

In another embodiment, the invention provides a method including a prediction of future heart rate values. The predicted heart rate value can be an average value, or a historical value.

For example, a trend value of the heart rate can be generated from previous measurements of the heart rate, and a standard deviation from the trend value can be determined. Where a measured heart rate value at a particular time falls outside of a range indicated by the trend value plus or minus a standard deviation, an indicator can be generated, notifying the user that a deviation from the predicted rate has occurred. Generally, deviations from predicted heart rate values are at least partially a negative representation of a users physical state.

In another example, two or more historical values can be mathematically utilized to determine a slope of the heart rate variability plot, the slope can be used to predict a future value plus or minus a deviation interval. Where a measured heart rate value at a particular time falls outside of a range indicated by the predicted value plus or minus a standard deviation, an indicator can be generated, notifying the user that a deviation from the predicted rate has occurred

In another embodiment, the indicator (kdev) can be a normalization factor for mathematical implementation into the HRV function as indicated in the following representation:

HRV=kdev*[(kALF)*LF]/[(kAHF)*HF].

Here, (kdev) is value between 0 and 1, and represents the consistency of the measured heart rate. For example, a very consistent heart rate might yield a deviation factor (kdev) of 1.0; whereas a highly inconsistent heart rate might yield a deviation factor (kdev) of 0.1.

One having skill in the art might apply less weight to the deviation factor by adjusting the value of (kdev) between 0.7 and 1.0, such that the resulting value of HRV is more adequately represented. In this example, it can be said that the deviation factor is weighted 30%, because the HRV value can be reduced by as much as 30%.

It is important to note that various mathematical representations can be generated by one having skill in the art in view of the aforementioned disclosure, however these variations will include the modification of prior art methods, i.e. the ratio of LF/HF, with a value mathematically representing the consistency (or inconsistency) of heart rate variations. It is therefore intended that the concept of modifying the LF/HF ratio with a mathematical indicator of the consistency of heart rate variations, no matter the mathematical representation thereof, be within the scope of the present invention.

Likewise, it is further intended that any mathematical manipulation of the prior art LF/HF ratio using one or more of an LF amplitude adjustment, or an HF amplitude adjustment, be within the scope of the present invention.

Accordingly, although one having skill in the art will immediately recognize a number of mathematical manipulations of the LF/HF ratio provided in the prior art, the description contained herein is provided to illustrate the various benefits of modifying the LF/HF ratio with one or more normalization factors as disclosed, for use with biofeedback representations, and the disclosure along with the following examples are not intended to limit the spirit and scope of the invention.

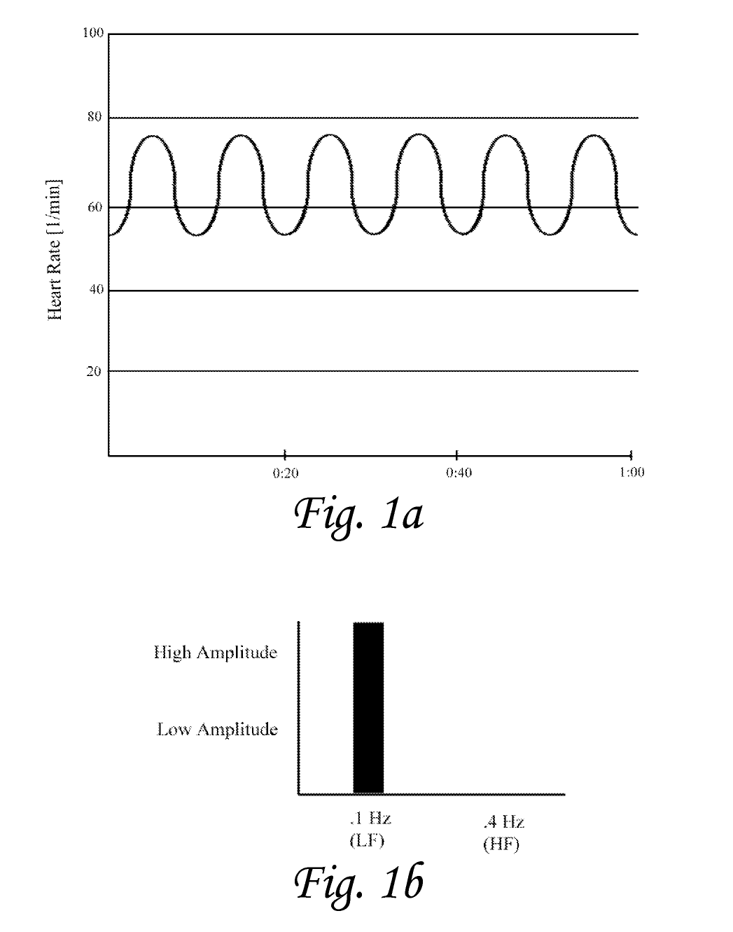

Referring now to FIG. 1, an illustrative representation of a spectrograph of an ideal HRV plot is provided. In the spectrograph, the measured heart rate varies from about 55 beats per minute to about 75 beats per minute. The HRV plot illustrates consistent LF variation without influence from HF variations, or noise.

The spectrograph of FIG. 1 is an ideal representation of a healthy user because there is consistency with the high amplitude LF variations in the heart rate. As mentioned previously, positive indicators of a user's relaxation state include: (i) a relatively high ratio of LF to HF variations in the user's heart rate; (ii) high amplitude LF variations in the heart rate, where high amplitude is about 10 beats per minute or more in variation; and (iii) consistency in the variations measured.

FIG. 1b indicates the high level of LF variations in the user's heart rate as indicated in FIG. 1a. Under prior art methods, the LF/HF ratio is at its maximum, because the LF variations of this spectrum dominate where there is no HF variability, or noise. Here, the prior art LF/HF methods would indicate the user's relaxation state as 100%. The user would be informed of the present relaxation state either by a numerical representation, or an illustrative representation as provided in FIG. 1b.

The methods of the present invention would further indicate that the particular user's relaxation state, as measured in FIG. 1a, is at a maximum, because the amplitude of LF variations is both consistent and relatively high (above 10 bpm).

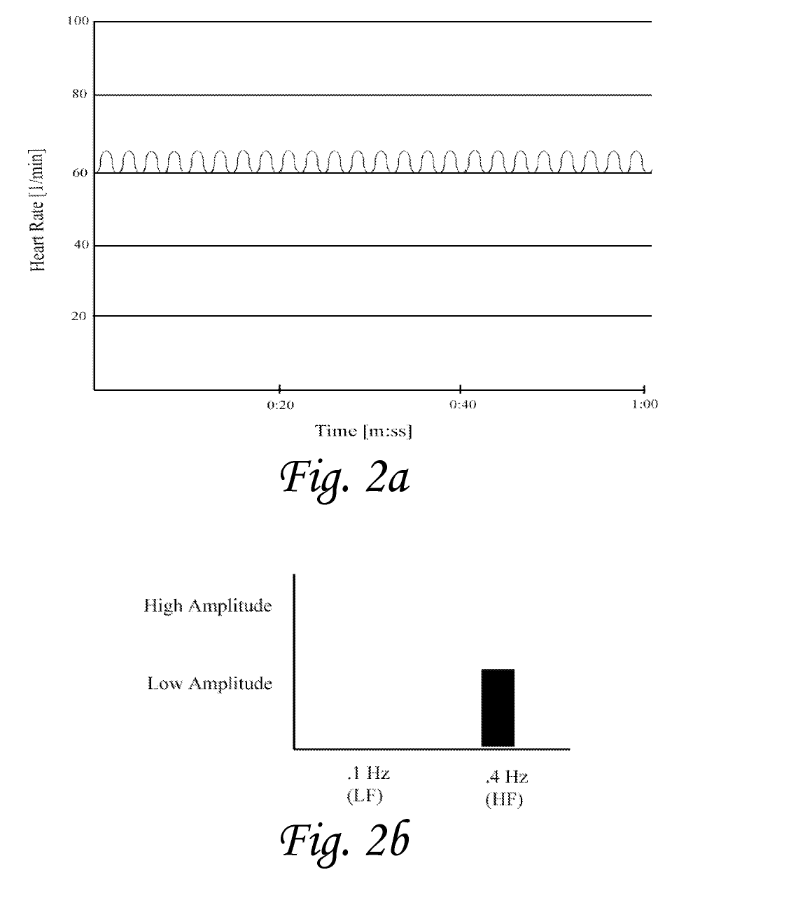

FIG. 2a represents a spectrograph of an HRV plot, where the heart rate varies from about 60 beats per minute to about 65 beats per minute. The HRV plot illustrates consistent HF variations in the heart rate, or noise. This type of a plot is theoretically very negative, and the only positive indicator is that the heart remains beating. However, for illustrative purposes, the plot of FIG. 2 is provided to illustrate a particular instance where high frequency variations in the heart rate dominate the spectrograph.

FIG. 2b indicates the high level of HF variations in the user's heart rate as indicated in FIG. 2a. Under prior art methods, the LF/HF ratio would be at its minimum, because the HF variations of this spectrum dominate where there is no LF variability. Here, the prior art LF/HF methods would indicate the users relaxation state as 0%. The user would be informed of the present relaxation state either by a numerical representation, or an illustrative representation as provided in FIG. 2b.

The methods of the present invention would further indicate that the particular users relaxation state, as illustrated in FIG. 2a, is at a minimum, because the plot lacks LF variability, and because the amplitude of HF variations is relatively high (about 5 bpm).

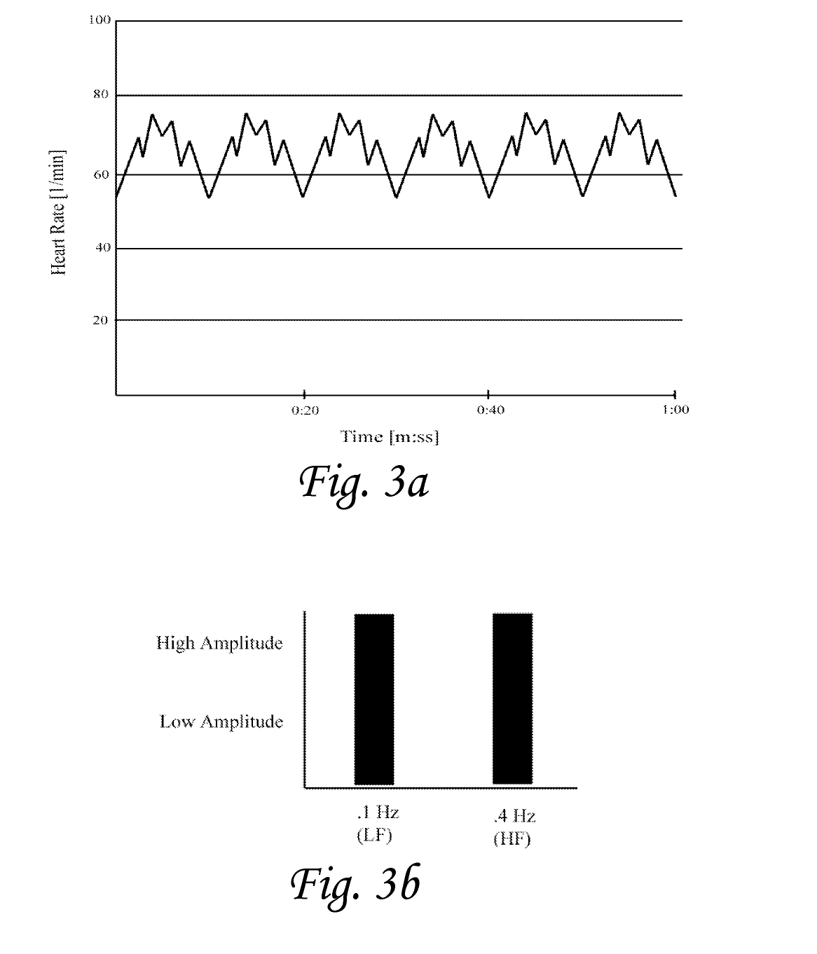

In the field, it is substantially more likely that a combination of LF and HF variations will be represented in a particular user's HRV plot. FIG. 3a illustrates a spectrograph of an HRV plot, where the heart rate varies from about 55 beats per minute to about 75 beats per minute. The HRV plot illustrates consistent LF variations in heart rate, with consistent influence from HF variations, or noise. In a practical sense, the user providing this type of spectrographic HRV representation is likely to be moderately stressed.

FIG. 3b is a graphical representation of the value of LF variations compared to the value of HF variations in the heart rate spectrograph of FIG. 3a. FIG. 3b further indicates the high level of LF variations in the heart rate, as well as the high level of HF variations in the heart rate. Under prior art methods for HRV analysis, the resulting value would be depicted as about 50%, because the high levels of HF will offset the equally high levels of LF, therefore placing the resulting HRV somewhere in the middle of the range, or about 50%.

The present invention can further analyze the HRV plot of FIG. 3a with respect to the LF amplitude, and HF amplitude. Here, although the levels of LF are high, they will be offset by the levels of HF, as well as the amplitude of the HF values. The extent for which the amplitude of the HF values reduces the end result is adjusted by changing the value of “k” for the HF amplitude factor (kAHF), where (0<k<1). Under the methods of the present invention, the resulting HRV will be less than 50%. Using these methods, the factors of HF amplitude and HF frequency combine to more adequately illustrate a level of moderate stress when compared to prior art methods.

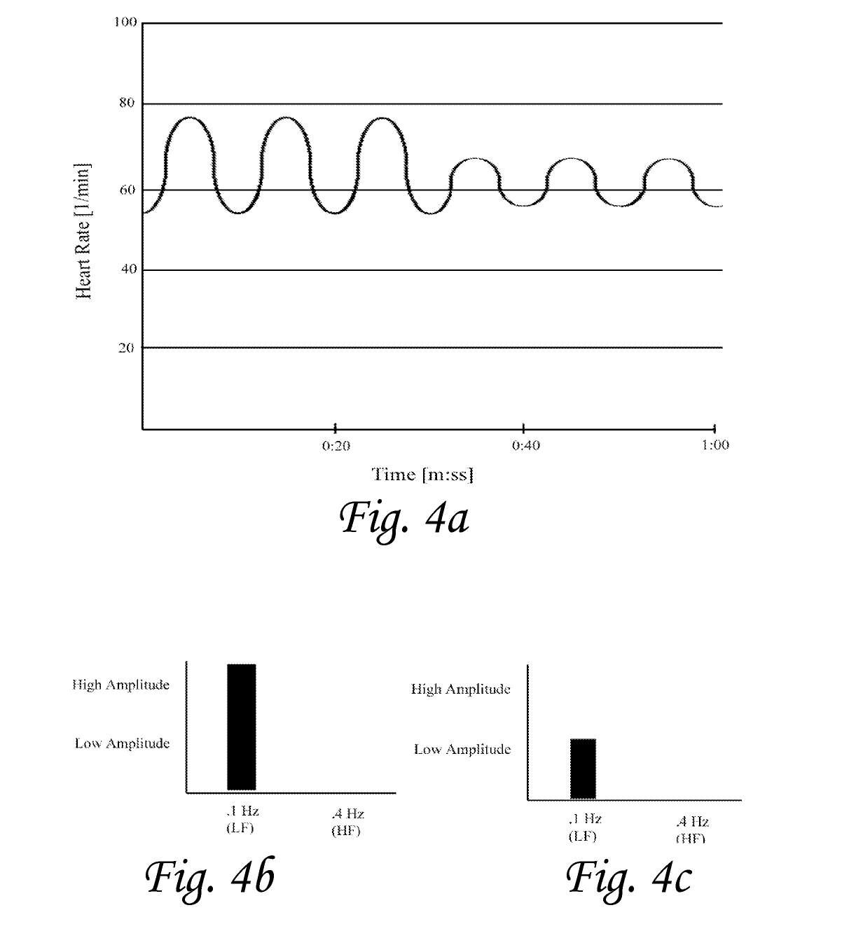

FIG. 4a is spectrograph of an HRV plot, where the heart rate varies from about 55 beats per minute (bpm) to about 75 beats per minute during the first 30 seconds of the period, and the heart rate varies from about 58 bpm to about 68 bpm in the last 30 seconds of the one minute period measured. The HRV plot illustrates consistent LF variation without influence from HF noise, however there is a decrease in the amplitude of LF variations over the second half of the measured period.

FIG. 4b is a graphical representation of the value of LF compared to the value of HF variations in the heart rate spectrograph of FIG. 4a during the first 30 seconds of the measured period.

FIG. 4c is a graphical representation of the value of LF compared to the value of HF variations in the heart rate spectrograph of FIG. 4a during the last 30 seconds of the measured period.

Under prior art methods for HRV analysis; i.e. the ration LF/HF, the resulting HRV would be depicted as about 100%, because the plot provides a high level of LF variations, and no HF variations in the heart rate. Therefore, the LF variations will dominate the analysis, resulting in about 100% resulting HRV value.

Utilizing the methods of the present invention, the decrease in amplitudes of the LF variations can be used to adjust the resulting HRV value to a more adequate representation of less than 100%. The LF amplitude factor (kALF), where (0<k<1), can be adjusted to weight the fluctuation in the amplitude with more or less influence. The present methods, therefore enable a method for HRV analysis that provides a more adequate representation of the HRV value, where a decrease in LF amplitude over a period of measurement no longer yields a maximum result of 100%. Accordingly, the present methods take into account the changes in LF, which are not adequately reflected by the LF/HF ratio of the prior art.

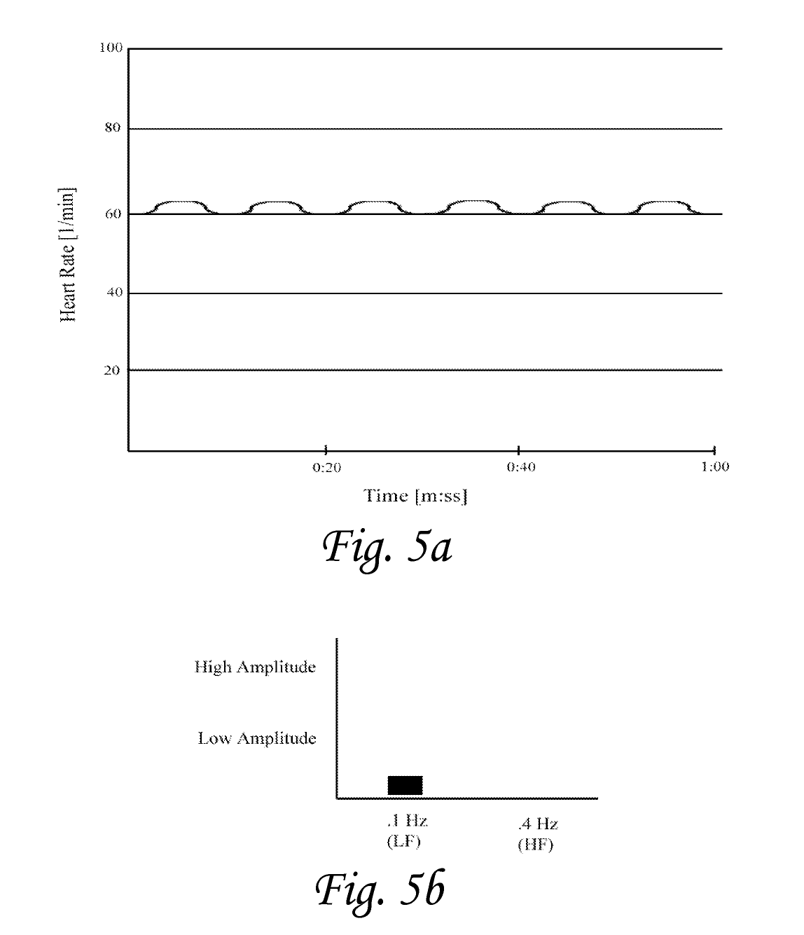

FIG. 5a is spectrograph of an HRV plot, where the heart rate varies from about 60 beats per minute to about 63 beats per minute. The HRV plot illustrates consistent LF variation without influence from HF noise, however the amplitude of LF variations is extremely low.

FIG. 5b is a graphical representation of the value of LF compared to the value of HF variations in the heart rate spectrograph of FIG. 5a.

Under prior art methods, the LF/HF ration would yield an HRV result of about 100%, because the levels of LF variations dominate in the absence of HF variations in the heart rate. In a practical sense, the plot of FIG. 5a is likely to resemble that of an elderly user, because variations in heart rate decrease with age. Here, the prior art methods would yield an HRV result of about 100%, even though the LF amplitudes are very weak.

In contrast, the methods of the present invention can be adapted to weight the LF amplitudes more or less to yield a more adequate result in the HRV representation. For example, here the high levels of LF will be offset by the LF Amplitude Factor (kALF), where (0<k<1), and the resulting HRV value would be significantly less than 100%. Accordingly, the present methods are an improvement over prior art methods, for at least the reason that the present methods are capable of illustrating relatively weak variations in the heart rate.

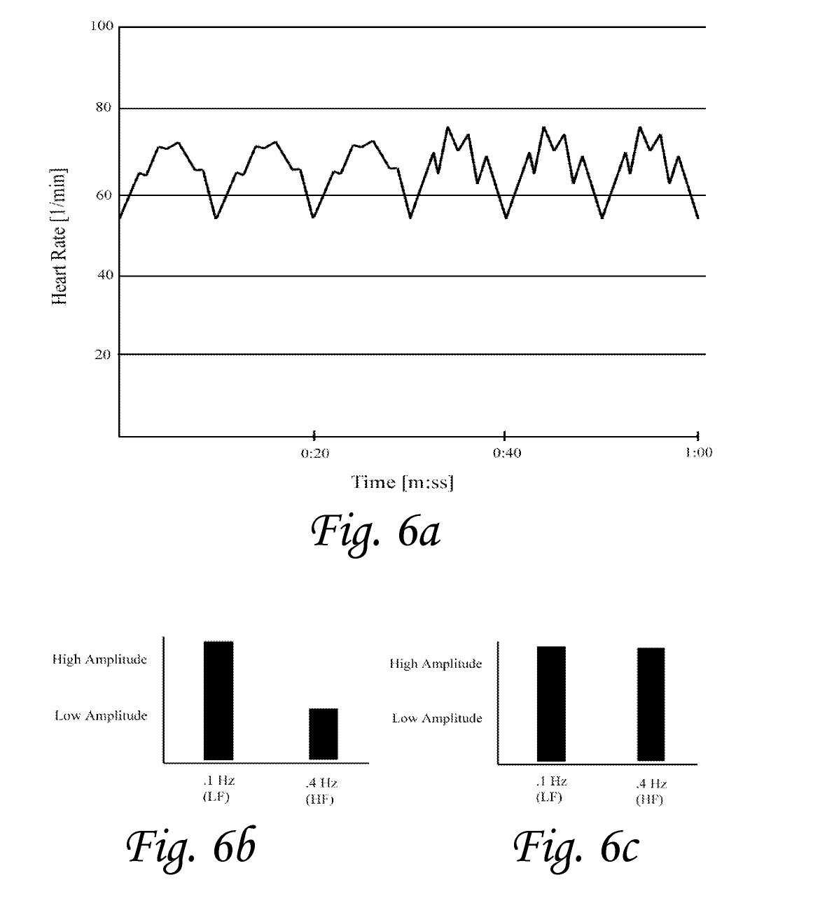

FIG. 6a is spectrograph of an HRV plot, where the heart rate varies from about 55 beats per minute to about 75 beats per minute. The HRV plot illustrates consistent LF variation with consistent influence from HF noise, however the HF noise is lower in amplitude during the first 30 seconds of the measured period, while the amplitude of the HF noise is more significant in the last 30 seconds of the period measured.

FIG. 6b is a graphical representation of the value of LF compared to the value of HF variations in the heart rate spectrograph of FIG. 6a during the first 30 seconds of the measured period.

FIG. 6c is a graphical representation of the value of LF compared to the value of HF variations in the heart rate spectrograph of FIG. 6a during the last 30 seconds of the measured period.

Under prior art methods for HRV analysis, the resulting HRV representation would be about 55% for the first 30 seconds of the period measured, and about 45% for the last 30 seconds for the period measured because the high level of LF variations will be offset by the high level of HF variations, therefore resulting in a middle range result, or about 50%. The prior art methods, however would not react to the change as rapidly as the present methods, therefore communication to the user would not be as efficient as the presently described methods.

Using the methods of the present invention, the HRV representation for the first 30 seconds of the measured period would yield a higher representation, in terms of percentage, than the last 30 seconds of the measured period. For example, during the first 30 seconds, the HRV plot illustrates a high level LF variations and a high level of HF variations, where the HF variations have a low amplitude. Accordingly, the first 30 seconds will yield an HRV representation of less than 100% and closer to 50%. However, the last 30 seconds of the interval provides an increase in the amplitude of HF variations, therefore the LF variations of the last 30 seconds will be offset with greater significance due to the increased HF amplitude during that interval. Accordingly, the HRV representation will yield about 60% for the first 30 seconds, and about 40% for the last 30 seconds of the one minute period measured, and illustrated, in FIG. 6a.

Additionally, in one embodiment of the invention where the application is a video representation used in biofeedback applications, certain attributes of the video representation are configured to vary with respect to a biofeedback indicator. In these embodiments, the measured period of FIG. 6a illustrates a practical example of how biofeedback indicators of the present invention can be useful in providing rapid communication of the present state or condition to a user. For example, where a color “Green” is assigned to represent an improvement, or decrease in the amount of HF variations in the heart rate, and the color “Red” is assigned to represent an increase in HF variations; the color at the 31st second of the measured period represented in FIG. 6a would switch from Green to Red. In another embodiment, a spectrum of colors from dark green, to light green, to yellow, to light red, and to dark red, is provided. As the measured level of HF changes, the spectrum of colors correspondingly changes. As described infra, other representations, such as speed, size, and other visual representations can be used to indicate changes in heart rate variability. Similarly, other indicators, such as the LF/HF ratio, LFadj./HFadj., changes in LF, changes in HF, and other indicators can be represented in a similar fashion. In this embodiment, a negative change in heart rate variability is always associated with a negative indicator, such as the “Red” color in the aforementioned example, regardless of the actual ratio of LF/HF.

Accordingly, the methods of the present invention more adequately represent the HRV by taking into consideration factors such as amplitude and frequency of heart rate variations. As discussed above, the LF amplitude factor, and HF amplitude factor, can be adjusted to more or less weight the significance of the corresponding amplitudes on the resulting HRV representation.

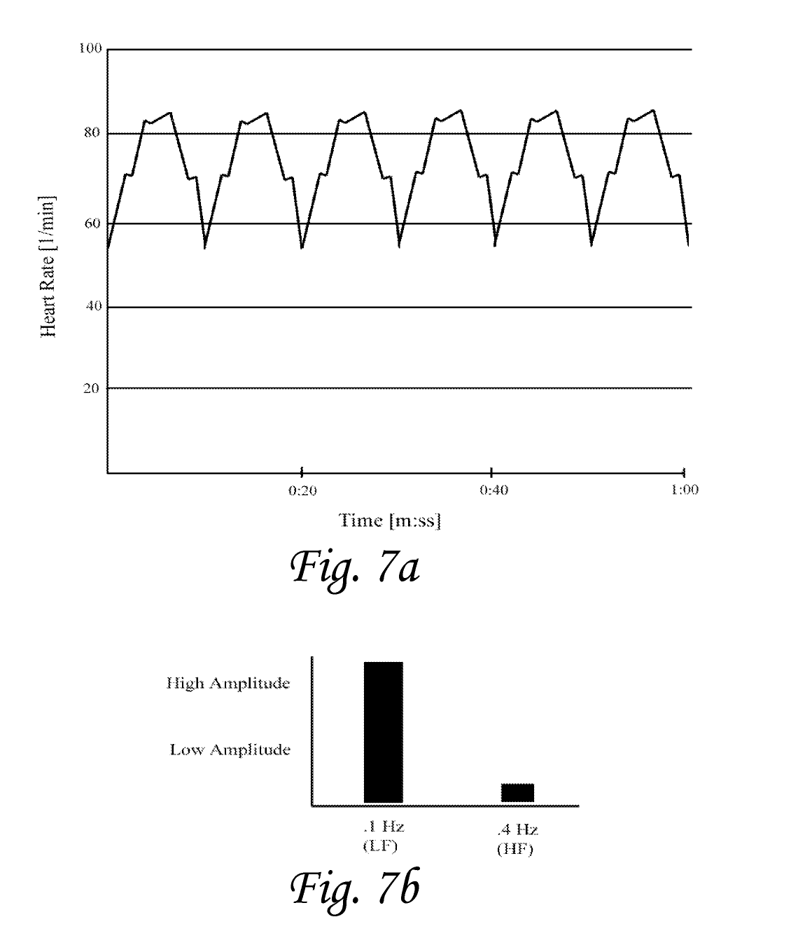

FIG. 7a is spectrograph of an HRV plot, where the heart rate varies from about 55 beats per minute to about 85 beats per minute. The HRV plot illustrates consistent LF variation with influence from HF noise, however the HF amplitude is relatively insignificant. The LF amplitude is very high, about 20 bpm, thereby offsetting the influence of the HF variations.

FIG. 7b is a graphical representation of the value of LF compared to the value of HF variations in the heart rate spectrograph of FIG. 7a.

Using prior art methods, the LF/HF ration would yield an HRV representation of about 75% because the high level of LF variations would be slightly offset by the level of HF variations. In contrast, the methods of the present invention would take into account the high amplitude of the LF variations, as well as the low amplitude of the HF variations, to modify the HRV result. Under the methods of the present invention, the HRV representation would yield about 90%, taking into consideration the amplitudes of LF and HF variations.

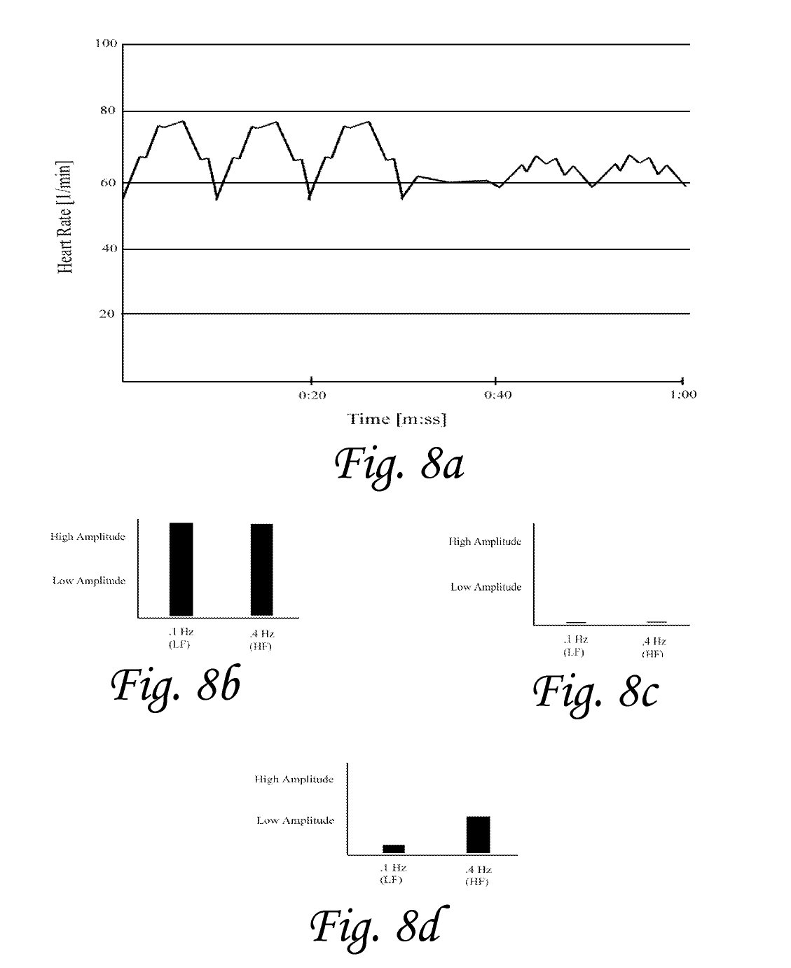

Another example of the improvements provided by the present inventive methods is illustrated when a user holds his or her breath, as illustrated in FIG. 8. FIG. 8a is spectrograph of an HRV plot, where the heart rate varies from about 55 beats per minute to about 75 beats per minute. The HRV plot illustrates consistent LF variation with influence from HF noise at a relatively minimal amplitude during the first 30 seconds of the measured period. The HRV is relatively flat between second 31 and second 40, while the user holds his or her breath. The HRV variations are then resumed at second 41 through the end of the measured interval, where lower amplitude LF variations combine with high amplitude HF variations.

FIG. 8b is a graphical representation of the value of LF compared to the value of HF variations in the heart rate spectrograph of FIG. 8a during the first 30 seconds of the measured period.

FIG. 8c is a graphical representation of the value of LF compared to the value of HF variations in the heart rate spectrograph of FIG. 8a during the ten second period starting at second 31 and ending at second 40 of the one-minute period for measurement.

FIG. 8d is a graphical representation of the value of LF compared to the value of HF variations in the heart rate spectrograph of FIG. 8a during the twenty second period starting at second 41 and ending at second 60 of the one-minute period for measurement.

Under prior art methods utilizing the LF/HF ratio, the first 30 seconds would yield an HRV representation of about 80%; seconds 31 through 40 would yield an HRV representation of about 80%, and the last 20 seconds of the measured period of FIG. 8a would yield an HRV representation of about 40%. It is important to note that seconds 31-40 would yield the same result as the first 30 seconds, even though the user is holding his or her breath. It is also important to note that under the prior art methods, these changes would not become apparent for a significant period of time, such as 30 seconds.

Using the methods of the present invention, the first 30 seconds of the period measured in FIG. 8a would yield an HRV representation of about 90% because the low amplitude of the HF variations would decrease the high levels of LF variations in the heart rate by a less significant amount. The period between second 31 and second 40 would yield an HRV closer to 0%, because there would be no LF or HF amplitude measured during this interval. The last 20 seconds of the period measured in FIG. 8a would yield an HRV representation of about 33% because the high amplitude of the HF variations would offset the low amplitude of the LF variations in the heart rate. FIG. 8 provides a practical example of how the present inventive methods more adequately represent the relaxation state of a user, taking into account the variations in LF and HF.

In another embodiment, with respect to heart rate variability illustrated in FIG. 8a, the method includes the assignment of a biofeedback representation; such as color, speed, size, and other visual representations, to a biofeedback indicator; such as LF/HF; LFadj./HFadj.; changes in LF; changes in HF; deviation from a predicted value of LF, HF, or the ration LF/HF; or other indicators of heart rate variability. For example, where speed is a positive indicator, the speed of an object in a biofeedback representation would be relatively fast during the first 30 seconds of the measured period illustrated in FIG. 8a. Seconds 31-40 would yield a very slow or stagnant object. At second 41, the object would increase in speed, however the speed would be relatively slow compared to the speed realized in the first 30 seconds of the measured period of FIG. 8a.

Therefore, although the present methods include improvements in the measured value of LF/HF, these methods can alternatively be used to measure and represent heart rate variability such as changes in LF, HF, or LF/HF in biofeedback applications, especially video games.

Therefore, although the present methods include improvements in the measured value of LF/HF, these methods can alternatively be used to measure and represent heart rate variability such as changes in LF, HF, or LF/HF in biofeedback applications, especially video games.

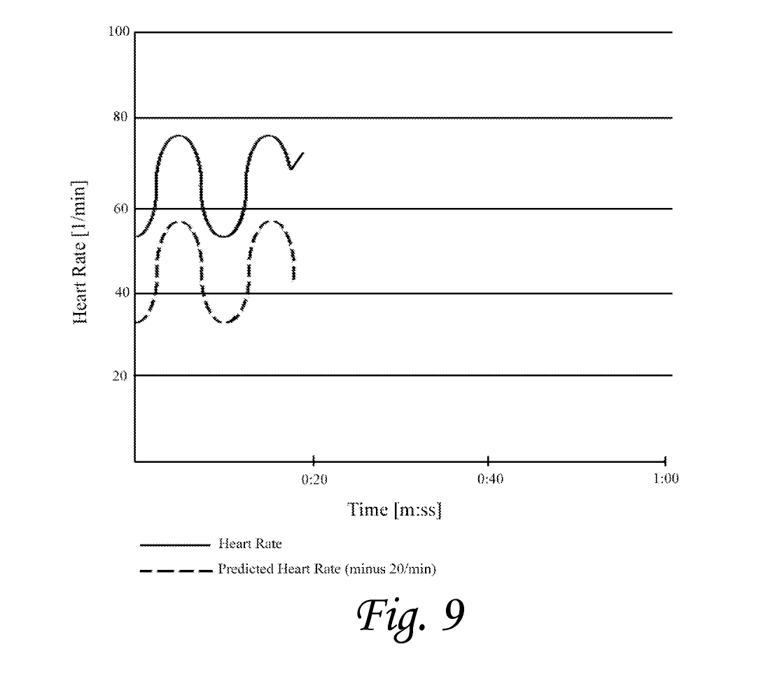

FIG. 9 is spectrograph of an HRV plot, where the heart rate varies from about 55 beats per minute to about 75 beats per minute. The HRV plot illustrates consistent LF variation without influence from HF noise; a second representation illustrates a predicted heart rate, where the measured heart rate deviates from the predicted heart rate at about 18 seconds into the measured period. For illustrative purposes, the predicted heart rate is illustrated below the measured values. To keep symmetry between the measured heart rate and the predicted heart rate, the predicted heart rate is provided at 20 bpm below the measured heart rate.

The inventor of the present invention recognized the benefits relating to immediate representation of HRV, and has contemplated the use of a predicted heart rate value. There are several ways to predict the heart rate value using historical data. One method includes taking a trend and standard deviation from historical data, then creating a notification where the measured heart rate lies outside of the range described by the trend value plus or minus the standard deviation. Another method includes defining a slope using two or more historical data points, and a standard deviation, and creating a notification where the measured heart rate lies outside the predicted heart rate as determined by the slope and standard deviation. The notification can be a normalization factor as described above and referred to as the deviation factor (kdev), where (0<kdev<1).

The methods of the present invention can include analysis of historical data to determine a trend, the trend including rhythmic increases and decreases in heart rate variability. Where the measured value deviates from the trend, a negative indicator can be represented to the user of a biofeedback program, or video game.

The deviation factor (kdev) can be weighted more or less to reflect the frequency of deviations from the predicted value. Because consistency in heart rate variation is a sign of a positive state of relaxation, deviations from the predicted rate can be interpreted as a negative representation of the relaxation state. Accordingly, as deviations from the predicted heart rate increase in frequency, the resulting HRV representation can be correlated in proportion. Additionally, the deviation factor (kdev) can be normalized to weight the HRV representation with more or less influence using methods described above as well as those apparent to one having skill in the art.

The HRV representations described above, including the LF/HF ratio; LFadj./HFadj., changes in LF, changes in HF, deviations from a trend value, and other HRV indicators, can be presented using numerical representations, for example “100%”, or alternatively the HRV representation can be illustrated as a bar graph; a line graph; a game using speed as the 0-100% value (higher percentage yields increased speed); a game where the characters shield, or other protective mechanism is controlled or influenced by the HRV value; a game where other characters interact with the user's character differently according the HRV value; a game where the user can perform certain tasks or tricks when the HRV value is sufficiently high; a game where the user acquires special abilities or powers where the HRV value is sufficiently high; a game where the user's abilities activate in various amounts based on the HRV value; a game where fog or visibility is reflected by the HRV value; or a coaching application where the coach uses the HRV value as part of the basis of its instructions or feedback to the user. Other representations of HRV values will become obvious to one having skill in the art in light of the foregoing examples.

Although the LF/HF is a useful indicator in biofeedback applications, the inventor of the present methods has recognized that a present change in the user's relaxation state is an important factor in Biofeedback software, and video game applications. These indicators of present changes in the user's relaxation state may include: the change in LF/HF; changes in HF amplitude or frequency; changes in LF amplitude or frequency; deviations from a trend value, where the trend value is LF/HF, HF amplitude, HF frequency, LF amplitude, LF frequency, or other indicators. These indicators can be used to immediately communicate changes, whether positive or negative, to a user, such that the user can adequately react to improve his or her relaxation state. As described above, these indicators can be linked to a biofeedback representation such as sound volume; sound speed; brightness of graphical display; contrast of graphical display; video game attributes such as: character speed, shields, color, interactions between characters, and other character changes.

Additionally, under the prior art LF/HF ratio, the heart rate variability is subject to a programmable maximum and minimum value; i.e. 100% and 0%, respectively. In biofeedback gaming applications, where there is a continuous need for feedback indicators, the prior art methods fail at each end of the spectrum, where feedback cannot be less than 0%, or more than 100%. Under the present inventive methods, a change in the users state is presented to the user in the form of a positive or negative indicator, therefore there is no limitation or terminal end of the spectrum because a continuous indicator is presented. In certain applications, the measured change in heart rate variability is just as useful, or even more useful than the traditional LF/HF indicator.

The foregoing description therefore provides methods for modifying the LF/HF ratio used in HRV analysis methods of the prior art. These modifications more adequately represent the state of relaxation, and immediately reflect changes in the HRV representation. Accordingly, the immediate feedback and more precise representations can enable a user to change his or her state, and therefore these methods are suitable for biofeedback applications such as behavioral training and video games.

The foregoing description of the embodiments of the present invention has been presented for purposed of illustration and description. It is not intended to be exhaustive or to limit the invention to the precise form disclosed. Obvious variations and modifications are possible in light of the above teachings. The embodiments set forth herein were chosen and described to provide the best illustration of the principles of the invention and its practical application to thereby enable one having skill in the art to utilize the invention in various embodiments.

What is claimed is:

1. A method for analysis of heart rate variability, comprising:
(i) providing an electrocardiographic device;
(ii) measuring a users heart rate over a period of time;
(iii) interpolating the value of low frequency (LF) variations and high frequency (HF) variations in the heart rate using one or more normalization factors selected from:
LF amplitude factor (kALF), HF amplitude factor (kAHF), and deviation factor (kdev);
(iv) representing the proportion of adjusted LF variations to adjusted HF variationsin the form of HRV=LFadj/HFadj.

2. The method of claim 1, wherein said period of time is sec⁻¹.
